(19) United States
(12) Patent Application Publication
(10) Pub. No.: US 2019/0298886 A1
(43) Pub. Date: Oct. 3, 2019

(54) Anti-Biofilm Osseointegrating Orthopedic Devices With Enhanced Elution Of Therapeutic Ions, And Methods Of Making And Using The Same

(71) Applicant: DiFusion, Inc., Austin, TX (US)

(72) Inventors: Joseph J. Crudden, Hudson, NH (US); Derrick Johns, Austin, TX (US); Sriram Sankar, Austin, TX (US)

(21) Appl. No.: 16/369,141

(22) Filed: Mar. 29, 2019

Related U.S. Application Data
(60) Provisional application No. 62/649,846, filed on Mar. 29, 2018.

(51) Int. Cl. A61L 27/54; A61L 27/56

(52) U.S. Cl. CPC A61L 27/54; A61L 27/56; B05D 1/12; A61L 2300/104; A61L 2420/02

(57) ABSTRACT

Anti-biofilm osseointegrating implantable devices that can elute therapeutic ions such as silver are provided by powder coating implantable substrates such as metal substrates. The polymer includes a polyarylether ketone such as PEEK, and zeolite, and the zeolite may be loaded with one or more therapeutic metal ions, such as silver, copper and/or zinc that exhibit antimicrobial properties. The devices, when implanted into a body and exposed to bodily fluid, may elute antimicrobial metal ions in a therapeutically effective amount.

This application claims priority of U.S. Provisional Application Ser. No. 62/649,846 filed Mar. 29, 2018, the disclosure of which is incorporated herein by reference in its entirety.

### BACKGROUND

Implantable medical devices are surgically implanted into the body for various reasons, including orthopedic applications (e.g., hip replacement, spinal procedures, knee replacement, bone fracture repair, etc.). In view of the structural integrity required by such devices, materials of fabrication are limited and generally consist of metal, plastic and composites.

The benefits derived from these devices are often offset by infection which in some cases can lead to sepsis and death. The most common organisms causing infections are Staphylococcus epidermidis and Staphylococcus aureus. Staphylococcus epidermidis is a major component of the normal bacterial flora of human skin and mucous membranes. It is a common pathogen that often colonizes patients in hospital settings who have surgical implants due to the microbes' ability to adhere to medical devices and form a biofilm. Additionally, methicillin-resistant Staphylococcus aureus (MRSA) is a type of staphylococcus bacteria that is resistant to many antibiotics is therefore of particular concern. Other gram-positive bacteria, gram-negative bacteria and fungal organisms also are causative organisms that may be problematic.

As microorganisms come in close proximity to the surface of the medical device, they will either be attracted or repelled by it depending on the sum of the different non-specific interactions. In biological systems, hydrophobic/hydrophilic interactions play an important role in the pathogenesis of a wide range of microbial infections.

Thermoplastic resins, including polyetherketoneketone (PEKK) and polyetheretherketone (PEEK) have been found to be a useful material for these implants. PEEK is particularly suitable because its modulus of elasticity closely matches that of bone.

It is also radiotranslucent. However, PEEK is a hydrophobic material, very resistant to permeation by liquids, and bacteria tend to adhere easily to these types of surfaces. It is also an organic material which does not carry significant surface charges. PEEK does not interact well with tissue, nor does it osseointegrate with bone. Indeed, PEEK implants present a smooth hydrophobic, uncharged, inert surface to surrounding tissue. These surfaces are not recognized as natural and become encapsulated by a fibrous apposition layer of soft tissue rather than becoming bonded to bone and tissue cells.

As a result, zeolite has been incorporated into PEEK to create a composite material with ceramic character that confers charge to the surface and renders it hydrophilic. Ceramics such as zeolite function as a cation cage, being able to be loaded with silver and other cations having antimicrobial properties. Metal zeolites can be used as an antimicrobial agent, such as by being mixed with the resins used as thermoplastic materials to make the implantable devices, or as coatings to be applied to the devices. The antimicrobial metal zeolites can be prepared by replacing all or part of the ion-exchangeable ions in zeolite with ammonium ions and antimicrobial metal ions. Such materials have been seen to perform extremely well in ovine and rabbit implant studies, showing high tissue compatibility, and bonding very well to bone and soft tissues alike.

Hip and knee implants have been very successful and provided a new lease on life for otherwise incapacitated patients. However, patients who have received metal hip and knee implants, particularly patients treated after tumor rescission, have been at risk of infection, aseptic loosening and in the worst cases, may need to have the limb amputated. Recent studies have shown that silver eluting hip stems can significantly improve outcomes, essentially eliminating the need for amputations. However, poorly controlled release of silver because of device design can result in the deleterious accumulation of excess silver in the joint over time. Release of silver and other therapeutic metal ions, from ion exchange ceramics such as zeolites, incorporated into polymer composites which are used to fabricate orthopaedic devices can provide for precision controlled release of the correct, safe and efficacious level of therapeutic ion.

Trauma plates and other forms of hardware are often used to repair broken bones, etc. If even a few bacteria attach to the surface of the repair device, lack of union, deep infection and even a failure of the surgical wound to close can ensue. Sometimes it may be necessary to transfer tissue such as muscle from another site, to bridge the surgical wound. Subsequently there may still be problems with recurrent infections from biofilm on the device, especially if an antibiotic resistant strain develops. Many repeat surgeries may be necessary, reducing the chance for a positive outcome.

Accordingly, it would be desirable to provide implantable medical devices with effective osseointegration ability and optional antimicrobial activity in order to reduce the growth of bacteria and risk of infection that do not suffer from the aforementioned drawbacks.

### SUMMARY

The shortcomings of the prior art have been overcome by embodiments disclosed herein, which relate to engineered anti-biofilm osseointegrating implantable biomaterial devices that can elute therapeutic ions such as silver. In certain embodiments, medical devices such as implants are engineered by powder coating a substrate with a polymer composite. The substrate may have the form of the desired implant, e.g., a hip stem, spinal spacer, skull flap, trauma plate, screw, rod etc. In certain embodiments, the implants are orthopedic implants, such as spinal, knee and hip implants, and are so shaped or configured. In some embodiments, the polymer includes a polyarylether ketone such as polyetherether ketone (PEEK). In some embodiments, the polymer also may include zeolite, and the zeolite optionally may be loaded with one or more therapeutic metal ions, such as silver, copper and/or zinc that exhibit antimicrobial properties when implanted into a body and exposed to bodily fluid or tissue. The devices, when implanted into a body and exposed to bodily fluid, when containing metal zeolite, may elute antimicrobial metal ions in a therapeutically effective amount. In certain embodiments, the source of antimicrobial activity includes ion-exchangeable cations contained in a zeolite. In certain embodiments, disclosed are methods of imparting antimicrobial activity to devices by controlling the delivery of certain cations through ion-exchange via a zeolite incorporated in the device introduced in a patient.

In some embodiments, the zeolite does not contain an antimicrobial metal ion, yet imparts hydrophilicity and a negative charge to the implant. This helps prevent biofilm formation and enhances osseointegration. In embodiments where antimicrobial ions are present, the PEEK/zeolite combination increases the ability of antimicrobial moieties to permeate in and kill the bacterial pathogen rather than be repelled by the hydrophobic surface properties of naked PEEK.

In certain embodiments, the device is configured for use in spinal fusion (arthrodesis) which is often employed to stabilize an unstable spinal column due to structural deformity, trauma, degeneration, etc. Fusion is a surgical technique in which one or more vertebrae of the spine are united together (“fused”) to reduce or eliminate relative motion between them or to fix the spatial relationship between them. Spinal fusions include posterolateral fusion, posterior lumbar interbody fusion, anterior lumbar interbody fusion, anterior/posterior spinal fusion, cervical fusion, thoracic fusion and interlaminar fusion. In certain embodiments, the devices are for insertion in an intervertebral space between adjacent vertebrae. In certain embodiments, a fusion site is identified between adjacent vertebrae and a bone graft is implanted at said site. In certain embodiments, the implant is a spinal interbody cage, including cages comprising titanium, carbon fibers, biocompatible materials such as polyetheretherketone (PEEK), polyetherketoneketone (PEKK), or other synthetic substances. In certain embodiments, zeolite particles are incorporated into the PEEK interbody cage. In certain embodiments, the cage is loaded with osseoconductive and/or osseoinductive agents to promote fusion. Preferably, the implant includes PEEK resin, and ceramic particles are incorporated into the resin such that a negative charge is imparted to an exposed surface of the resin. The term “exposed surface” is intended to include one or more surfaces of an implantable device that when implanted, is exposed to or in contact with body tissue and/or fluids.

In certain embodiments, a method of powder coating an implantable substrate is set forth, comprising forming a polymer formulation comprising a mixture of a polymer resin and zeolite; providing an implantable substrate; spray coating the polymer formulation onto the substrate; and curing the coating on the substrate. In some embodiments, the zeolite may comprise antimicrobial metal ions such as silver ions. In some embodiments, the substrate is shaped as a hip stem, knee implant, trauma plate or intervertebral spacer. In some embodiments, the formulation is a blend of zeolite powder and polymer resin powder. In some embodiments, the blend may include a micronized porogen, which is subsequently extracted to leave a network of pores that expose the zeolite.

The hydrophilicity imparted by the zeolite results in an engineered biomaterial that interacts with the bone of the patient and induces a bone/biomaterial fusion. The presence of the zeolite also results in a rapid transition from M1 proinflammatory macrophage phenotype to the M2 macrophage phenotype, thereby minimizing fibrous encapsulation and facilitating the deposition of cite appropriate tissue ultimately yielding constructive and functional tissue remodeling. The negative charge imparted by the zeolite attracts and adheres the required precursor proteins for bone growth to the implant surface, and ultimately supports long term osseointegration.

In some embodiments, A method of promoting osseointegration of an implant in a patient is disclosed, comprising: blending zeolite, a micronized porogen and a polyetheretherketone resin to form a polymer formulation, wherein the zeolite is blended into the resin in an amount effective to render the polyetheretherketone resin hydrophilic and impart a negative charge to said polyetheretherketone resin; spray coating the polymer formulation onto an implant substrate; curing the coating on the substrate; extracting the porogen to form a network of pores in the coating; and implanting the resulting implant into a patient in need thereof.

In some embodiments, a surgical implant is disclosed, comprising: a metal substrate; a spray-applied coating on the substrate, the spray-applied coating comprising polyetheretherketone and zeolite, wherein the coating has a network of pores formed by extraction of a micronized porogen from a blend of the polyetheretherketone, zeolite and micronized porogen after being spray-applied onto the metal substrate.

### BRIEF DESCRIPTION OF THE DRAWINGS

The embodiments disclosed herein may take form in various components and arrangements of components, and in various process operations and arrangements of process operations. The drawings are only for purposes of illustrating preferred embodiments and are not to be construed as limiting.

FIG. 1 is a photograph of a powder coated hip stem and washer cup, which attaches to the pelvis, in accordance with certain embodiments;

FIG. 2 is a photomicrograph of a porous polymer in accordance with certain embodiments;

FIG. 3 is another photomricrograph of a porous polymer in accordance with certain embodiments; and

FIG. 4 is a macrophotograph of the surface of a coated substrate in accordance with certain embodiments.

### DETAILED DESCRIPTION

A more complete understanding of the components, processes, systems and apparatuses disclosed herein can be obtained by reference to the accompanying drawings. The figures are merely schematic representations based on convenience and the ease of demonstrating the present disclosure, and are, therefore, not necessarily intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings. In the drawings and the following description below, it is to be understood that like numeric designations refer to components of like function.

The singular forms “a,” “an,” and “the” include plural referents unless the context clearly dictates otherwise.

As used in the specification, various devices and parts may be described as “comprising” other components. The terms “comprise(s),” “include(s),” “having,” “has,” “can,” “contain(s),” and variants thereof, as used herein, are intended to be open-ended transitional phrases, terms, or words that do not preclude the possibility of additional components.

All ranges disclosed herein are inclusive of the recited endpoint and independently combinable (for example, the range of “from 2 inches to 10 inches” is inclusive of the endpoints, 2 inches and 10 inches, and all the intermediate values).

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. The modifier “about” should also be considered as disclosing the range defined by the absolute values of the two endpoints. For example, the expression “from about 2 to about 4” also discloses the range “from 2 to 4.”

It should be noted that many of the terms used herein are relative terms. For example, the terms “upper” and “lower” are relative to each other in location, i.e. an upper component is located at a higher elevation than a lower component, and should not be construed as requiring a particular orientation or location of the structure. As a further example, the terms “interior”, “exterior”, “inward”, and “outward” are relative to a center, and should not be construed as requiring a particular orientation or location of the structure.

The terms “top” and “bottom” are relative to an absolute reference, i.e. the surface of the earth. Put another way, a top location is always located at a higher elevation than a bottom location, toward the surface of the earth.

Certain embodiments relate to a biomaterial formulated by blending a base polymer, preferably PEEK, with a negatively charged zeolite, and spray coating the blend onto an implant substrate. The zeolite changes the surface topography, charging characteristics, and pH of the resulting composite in a predictable, suitable manner for the surgical environment and long-term healing of the patient into which the device is implanted. Attributes imparted by the zeolite include bone fusion, biocompatibility, negative charge, hydrophilicity and osseoconductivity. Attributes provided by the PEEK base polymer include radiolucency, biocompatibility, durability and versatility. The resulting composite blend provides a uniform material construct and excellent workability.

Particularly compelling is the ability of the zeolite to reduce or eliminate the immune response that is generated when naked PEEK is implanted. It is a well-recognized problem that the human immune system reacts to the presence of naked PEEK as a foreign, unnatural substance, and as a damage/danger associated molecular pattern (DAMP). Consequently, the human body responds to the presence of naked PEEK by encapsulating it, causing bone resorption, and initiating a pain response. This is believed to be directly related to the hydrophobic, uncharged and water repellant nature of naked PEEK. Adding zeolite to the PEEK polymer increases proliferation, differentiation and transforms growth factor beta production in normal adult human osteoblast-like cells. The hydrophilic surface of the resulting implant down-regulates pro-inflammatory cytokines interleukin 1 & 6, which modulates the immune response, facilitates the enhanced bone would healing and osseointegration, allows for early cell adhesion and ultimate osseoconduction, and reduces pain. IL1-Beta upregulates inflammatory immune-response, and IL6-Beta haw been shown to have a direct relation to spinal disc pain. Both have been shown to down regulate osteoblast cells while up-regulating osteoclast cells, showing the increased fibrosis and resorption of bone with which naked hydrophobic PEEK has been well associated.

Accordingly, embodiments disclosed herein relate to medical implants and methods of making the same, that may include the use of ceramics, preferably zeolites, to impart hydrophilicity to a hydrophobic polymer base, and in some embodiments function as a cationic cage to deliver and dose one or more antimicrobial cations. Suitable cations include silver, copper, zinc, mercury, tin, lead, gold, bismuth, cadmium, chromium, thallium ions and mixtures thereof, with silver, zinc and/or copper being preferred, and silver being especially preferred. In certain embodiments, the implants are load-bearing surgical implants.

In some embodiments, either natural zeolites or synthetic zeolites can be used to make the zeolites used in the embodiments disclosed herein. “Zeolite” is an aluminosilicate having a three-dimensional skeletal structure that is represented by the formula: XM_{2/n}O.Al₂O₃.YsiO₂.ZH₂O, wherein M represents an ion-exchangeable ion, generally a monovalent or divalent metal ion, n represents the atomic valency of the (metal) ion, X and Y represent coefficients of metal oxide and silica respectively, and Z represents the number of water of crystallization. Examples of such zeolites include A-type zeolites, X-type zeolites, Y-type zeolites, T-type zeolites, high-silica zeolites, sodalite, mordenite, analcite, clinoptilolite, chabazite and erionite. A-type zeolites are particularly preferred, such as 4 A zeolite having particle size ranges from 1 to 10 microns with a narrow distribution of 4 microns.

In certain embodiments, zeolite in powder form, such as 4 micron zeolite, with or without incorporated metal ions, may be blended with a fine powder of the thermoplastic resin such as PEEK (commercially available from Solvay, Ketaspire-121FP), such as in a rotating drum. In some embodiments the powders are blended to homogeneity. In some embodiments, a micronized porogen may be included in the blend as discussed in greater detail below. The resulting powder formulation may be sprayed onto the implant substrate, such as with an electrostatic coater. In other embodiments, the zeolite, with or without incorporated metal ions, and with or without a porogen, is blended into molten thermoplastic resin, and then the resulting hardened composite (e.g., in pellet form) is ground into a powder and sprayed onto the implant substrate, such as with an electrostatic coater.

In some embodiments, a slurry or aqueous dispersion of the thermoplastic resin (e.g., PEEK powder) and zeolite, with or without metal ions incorporated therein, can be formed and used for coating an implant substrate. In certain embodiments, the slurry or aqueous dispersion is continuously mixed. Additional materials can be added to the mixture, including a porogen to impart porosity to the coating as discussed in greater detail below.

In some embodiments, the implant substrate is metal, and is heated to a temperature sufficient to evaporate water present in the coating formulation once the coating is spray coated on the substrate. Suitable temperatures are about 100° C. or higher, for example.

In some embodiments, the powder formulation may be flame sprayed onto the implant substrate. In this process, the combustion of a gas/oxygen mixture generates the thermal energy to melt the powder blend and warm the surface of the implant substrate. A carrier gas, such as compressed air, may serve as the medium for transporting the melted particles to the substrate surface.

Zeolites can be incorporated into masterbatches of a range of polymers. For final incorporation into the resin, a masterbatch can be produced by incorporating typically about 20% zeolite. When provided in this form, the pellets of masterbatch PEEK containing the zeolite particles can be further reduced by mixing with more virgin resin (e.g., PEEK) such as at high temperature and under high shear. The resulting pellets can be ground or otherwise formed into powder for subsequent coating on a substrate such as with an electrostatic coater.

Other suitable resins include low density polyethylene, polypropylene, ultra-high molecular weight polyethylene or polystyrene, polyvinyl chloride, ABS resins, silicones, rubber, and mixtures thereof, and reinforced resins, such as ceramic or carbon fiber-reinforced resins, particularly carbon fiber-reinforced PEEK. The latter can be produced by dispersing the reinforcing material or materials (e.g., carbon fibers) in the polymer matrix, such as by twin screw compounding of implantable PEEK polymer with carbon fibers. The resulting carbon fiber-reinforced product can be used to direct injection mold final devices and near net shapes, or it can be extruded into stock shapes for machining. The incorporation of fibers or other suitable reinforcing material(s) provides high wear resistance, a Young's modulus of 12 GPa (matching the modulus of cortical bone) and providing sufficient strength to permit its use in very thin implant designs which distribute the stress more efficiently to the bone. The amount of reinforcing material such as carbon fiber incorporated into the resin such as PEEK can be varied, such as to modify the Young's modulus and flexural strength. One suitable amount is 30 wt % carbon fiber.

In certain embodiments, metal substrates that are formed, shaped or configured as an implant, such as an orthopedic implant such as a hip stem, intervertebral spacer or trauma plate, are powder coated with a mixture including polymer and zeolite, and preferably polymer, zeolite and a porogen. Powder coating is a potentially solvent-free process which can be used to apply a 100% solids, polymer or polymer composite coating to materials such as orthopedic implant substrates which may be fabricated from metals such as stainless steel, titanium or suitable metal alloys. Powder coating functions by applying a potential difference between the object to be coated and the nozzle of a powder coating gun from which fine particles of coating material are blown using a driving force such as compressed air, for example. The particles pick up a charge which is opposite to the charge on the target object and, once expelled from the nozzle, are drawn by the charge difference to the surface of the target object. The powder uniformly coats the exposed surface of the target object, with a slight preferential attraction to edges and corners because of the increased charges present there. The object may be then carefully moved to a curing oven or furnace which is raised to a sufficient temperature to fuse the coating of powder particles and to allow it to flow and form a strong tenacious film which is firmly bonded to the surface of the object. Once the temperature drops below the fusion temperature of the coating, the coating becomes completely stable.

In certain embodiments, a powder formulation is made for powder coating onto a substrate, such as a metal substrate suitable for implanting. The powder formulation may include the polymer, such as PEEK, and zeolite, which may be loaded with a metal cation either before the zeolite is added to the polymer or post-loaded into the fused coating after it is applied to the device (or after blending with the polymer). It may also include a porogen. In certain embodiments, the metal cation, if present, is present at a level below the ion-exchange capacity in at least a portion of the zeolite particles. In some embodiments, a suitable amount of the zeolite is thoroughly mixed with a suitable amount of polymer, such as with a drum roller. In some embodiments, the amount of zeolite mixed with the polymer may range from 0.01 to 50 wt. %, more preferably 15% to 20 wt. %.

Where an antimicrobial effect is desired, the amount of metal ions in the zeolite should be sufficient such that they are present in an antimicrobial effective amount when implanted into the body of a patient. For example, suitable amounts can range from about 0.1 to about 20 or 30% of the exposed zeolite (w/w %). These levels can be determined by complete extraction and determination of metal ion concentration in the extraction solution by Inductively Coupled Plasma Optical Emission Spectroscopy, ICPOES, or atomic absorption. Preferably the ion-exchanged antimicrobial metal cations are present at a level less than the ion-exchange capacity of the ceramic particles. The amount of ammonium ions is preferably limited to from about 0.5 to about 15 wt. %, more preferably 1.5 to 5 wt. %. For applications where strength is not of the utmost importance the loading of zeolite can be taken as high as 50%. At such loadings the permeation of metal ions can permeate well below the surface layer due to interparticle contact, and much greater loadings of metal ions are possible.

In some embodiments, the polymer powder formulation can be formed by adding zeolite that is devoid of metal ions, and then post-loading the zeolite with metal ions after the powder has been spray applied to the substrate and cooled to between 0 and 100° C., preferably cooled to room temperature. By incorporating the metal cation(s) into the zeolite after the blend has cooled, deleterious oxidation of the metal ions is reduced or eliminated. Metal ion salt solutions, such as nitrates, acetates, benzoates, carbonates, oxides, etc., can be used to accomplish this. Addition of nitric acid to the infusion solution also may be advantageous in that it can etch the surface of the implant, providing additional surface area for ion exchange. That is, the zeolite may be charged with metal ions at a temperature between about 0 and 100° C., preferably about room temperature) from a metal ion source such as an aqueous metal ion solution, such as silver nitrate, copper nitrate and zinc nitrate, alone or in combination. Cooling to lower temperatures gives lower loading rates but higher stability. Loading at even higher temperatures can be carried out at a faster rate by maintaining the system under pressure, such as in a pressure cooker or autoclave. The content of the ions can be controlled by adjusting the concentration of each ion species (or salt) in the solution.

For example, the PEEK zeolite composite (which may include a porogen) can be loaded by bringing the material into contact with an aqueous mixed solution containing ammonium ions and antimicrobial metal ions such as silver copper, zinc etc. The most suitable temperatures at which the infusion can be carried out range from 5° C. to 75° C., but higher temperatures may also be used even above 100° C. if the reaction vessel is held under pressure. Higher temperatures will show increased infusion rates, but lower temperatures may eventually produce more uniform and higher loadings. The pH of the infusion solution can range from about 2 to about 11 but is preferably from about 4 to about 7.

Suitable sources of ammonium ions include ammonium nitrate, ammonium sulfate and ammonium acetate. Suitable sources of the antimicrobial metal ions include: a silver ion source such as silver nitrate, silver sulfate, silver perchlorate, silver acetate, diamine silver nitrate and diamine silver nitrate; a copper ion source such as copper(II) nitrate, copper sulfate, copper perchlorate, copper acetate, tetracyan copper potassium; a zinc ion source such as zinc(II) nitrate, zinc sulfate, zinc perchlorate, zinc acetate and zinc thiocyanate.

Suitable thicknesses of the coating on the substrate may range from about 10 to about 50 microns or thicker, depending on the application. Multiple coatings can be applied. Preferably a PEEK coating containing no additives is applied as a base layer followed by a layer containing zeolite (with or without metal cations incorporated therein) and optionally an added porogen such as micronized sodium chloride salt. In this way, any potential for corrosion at the surface of the implant may be mitigated. In some embodiments, layers of coatings can be applied that have different amounts of zeolite. In some embodiments, a gradient of zeolite amounts in the resin is created, such as layers beginning at 0% zeolite at the substrate surface, and increasing amounts of zeolite as the layers progress to the final outer layer. The thickness of the coating can be uniform over the entire surface of the substrate, or can be non-uniform.

In some embodiments, a porogen may be added to impart porosity or to incorporate channels and/or voids to the polymer in an effort to allow bony in-growth into the surface of the implant. Complete porosity (an open pore structure) can be expected at 50 (volume/volume) %. For non-load bearing surfaces up to 50% salt may be used, producing an open cell structure which is completely permeable and accessible to adjacent tissue and fluids. Furthermore, the porosity allows the delivery of therapeutic agents from throughout the structure of the device and allows bone and tissue to grow deep into and through the device structure. In certain embodiments, the soluble salts are thoroughly and completely washed from the matrix with pure water. Suitable porogens include sodium chloride, sodium citrate, sodium tartrate, potassium chloride, sodium fluoride, potassium fluoride, sodium iodide, sodium nitrate, sodium sulphate, sodium iodate, and mixtures thereof. Residual exposed salt can be washed from the surface of the implant using pure water. Preferably the porogen is micronized salt, preferably sodium chloride, having an average particle size ranging from 2 to 10 microns, more preferably from 4 to 8 microns. In some embodiments, the size of the porogen is similar to or about the same as the size of the zeolite particles. Micronizing the porogen allows for the powder mixture to remain homogeneous through the powder handling steps of the process, and allows uniform pore distribution in the resulting composite. Suitable amounts of porogen include from about 2 to about 50% by weight of the composite blend, more preferably from about 5 to about 20% by weight. The result is a tortuous path within the pore network that will result in a much-enhanced capability for the device to carry exchanged ions as well as providing more prolonged release kinetics.

Such an open exposed structure produced with porogen and unloaded zeolite may be post-loaded with therapeutic metal ions and allows for a wide scope for tuning levels of release of ions once the implant is surgically placed in a patient.

In certain embodiments, a blend of micronized porogen (e.g., micronized sodium chloride), 4 A zeolite and PEEK can be extruded into pellets which can be subsequently extruded into PEEK composite rods by conventional techniques for subsequent machining into orthopedic devices such as implants, rods, screws, plates etc. After machining, the porogen can be extracted with aqueous solution to develop the porosity in the composite and the exposed zeolite both on the surface and within the exposed pores optionally can be post-loaded with therapeutic metal ions such as silver, zinc, copper, magnesium, strontium etc., such as at room temperature. Since the metal cations are not exposed to the high process temperatures (e.g., 300-400° C. necessarily to render the PEEK molten and incorporate the zeolite, or the high temperatures used during spray application of the coating), deleterious oxidation of the metal cations does not occur.

The powder combination described above can also be injection molded at high temperature, e.g., 300 to 400° C., as is, or after being extruded into pellets, into, for instance, orthopedic implants, screws, plates, rods, flaps, catheters or fixtures.

The resulting device may be introduced into the body surgically. The rate of release of antimicrobial metal ions, when present, is governed by the extent of loading of the polymer with zeolite and the extent to which the exposed zeolite is charged with metal ions. The electrolyte concentration in blood and body fluids is relatively constant and will cause ion exchange with ions such as silver, copper and zinc, etc. from the surface of the implant, which deactivate or kill gram positive and gram-negative organisms, including E. coli and Staphylococcus aureus. Effective antimicrobial control (e.g., a six-log reduction of microorganisms) is achieved even at low metal ion concentrations of 40 ppb.

### Example 1

PEEK powder, supplied by Solvay, about 10 micron particle size diameter, was carefully weighted out. Silver zeolite, 4 A Zeolite loaded to 22-24% silver, of about 4 micron particle size, was added to the PEEK powder in a container, and both materials were mixed thoroughly by rotating the container on a drum roller for about 10 minutes at room temperature.

| | Gm | (w/w) % |
|---|---|---|
| Peek Powder | 170 | 85.0 |
| Silver Zeolite | 30 | 15.0 |
| Total | 200 | 100.0 |

The resulting composite mixture was added to an Eastman 1676 hotcoat powder coating gun. This is an Eastwood Dual Voltage HotCoat Powder Coating Gun that can provide the most complete coverage of a target object, ranging from tight areas to large surfaces, allowing powder coating of interiors and hard-to-reach corners with ease. HotCoat powder coating requires a 5-10 psi compressed air source and a dedicated electric oven or toaster oven.

A degreased and lightly sanded metal hip stem and cup, supplied by Marle Industries, was suspended on a metal frame in a fume hood and the system, masked where appropriate with aluminum foil, was set up as described in the operating instructions of the Eastman gun. A clip was attached to the suspending frame close to the pieces to be coated. The powder coating gun was connected to the main power and to an air compressor as described in the gun instructions. The button to activate the charging mechanism was depressed and subsequently the gun trigger was pulled. A fine mist of powder was blown from the gun and was drawn to and encapsulated on the hip stem and cup. The objects could be clearly seen to be well coated even on the edges and corners except where parts of the surface were masked with aluminum foil.

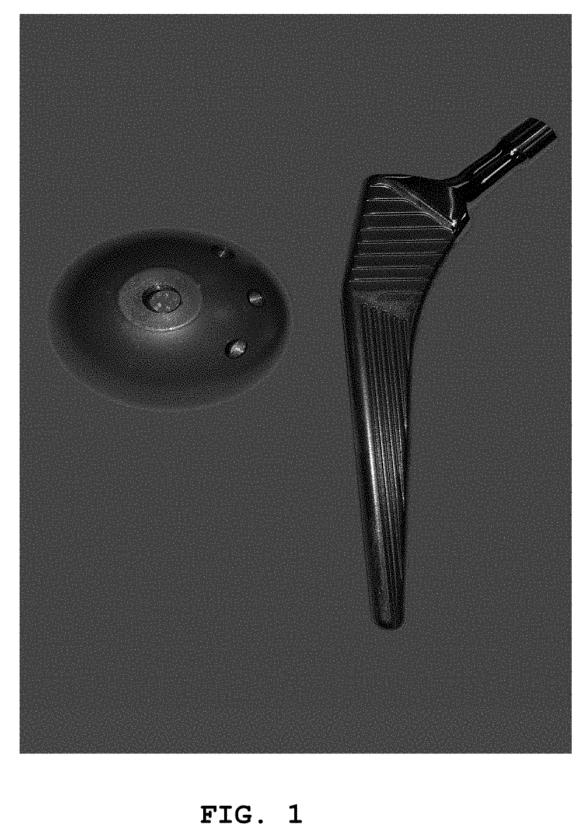

The objects and suspending frame were carefully moved to a 16 cubic foot ceramic kiln (Cress Turbofire) which was pre-heated to about 385° C., and allowed to heat and cure for about 10 minutes. Then the objects were removed from the furnace and allowed to cool. The coating cured to a tough semitransparent chocolate colored film which was strongly bonded to the metal surfaces of the hip stem and cup, as seen in FIG. 1.

### Example 2

A steel washer of about 1.5 inches diameter was coated with the same material of Example 1 using the same procedure. The washer with the applied powder coating was heated and the film cured in a laboratory furnace, Labline, for about 10 minutes at 385 to 400° C. A similar result as in Example 1 was achieved.

### Example 3

### Determination of Silver Elution Rate.

The washer from Example 2 was placed in a non-venting petri dish containing 50 ml of 0.9% sodium nitrate solution and placed on a Labline laboratory rocker and allowed to gently rock for 22 hours. After 22 hours, a sample of eluent was removed from the Petri dish and assayed for silver using TCP OES. The solution was found to contain 530 ppb silver. This is an excellent level effective to provide therapeutic antimicrobial efficacy while being safe to human tissue.

### Example 4

Conventional table salt was micronized to a very fine particle size in a coffee mill. The salt was then incorporated into a polymer resin and metal zeolite formulation to determine if it could produce a surface which would be more conducive to bony ingrowth and keying of the bone into the surface of the implants.

Composition

| Component | gm | (w/w) % |
|---|---|---|
| Peek Powder | 85 | 47.5 |
| Sodium chloride (Micronized) | 85 | 47.5 |
| Silver Zeolite | 30 | 15.0 |
| Total | 200 | 100.0 |

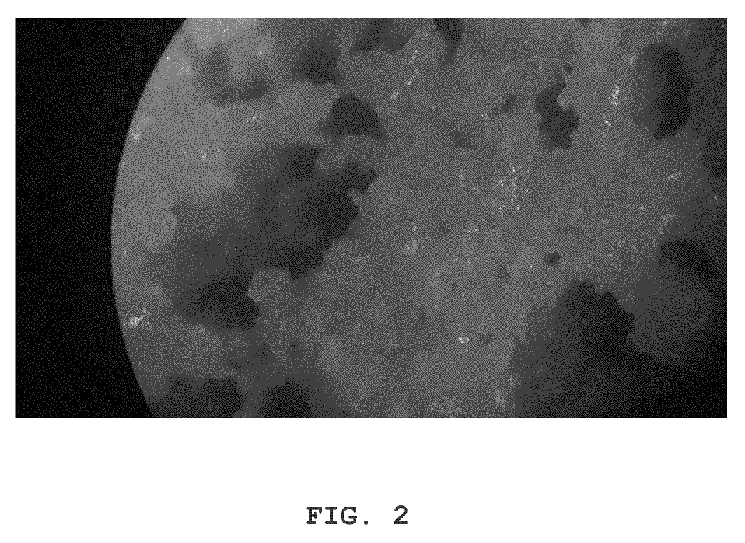

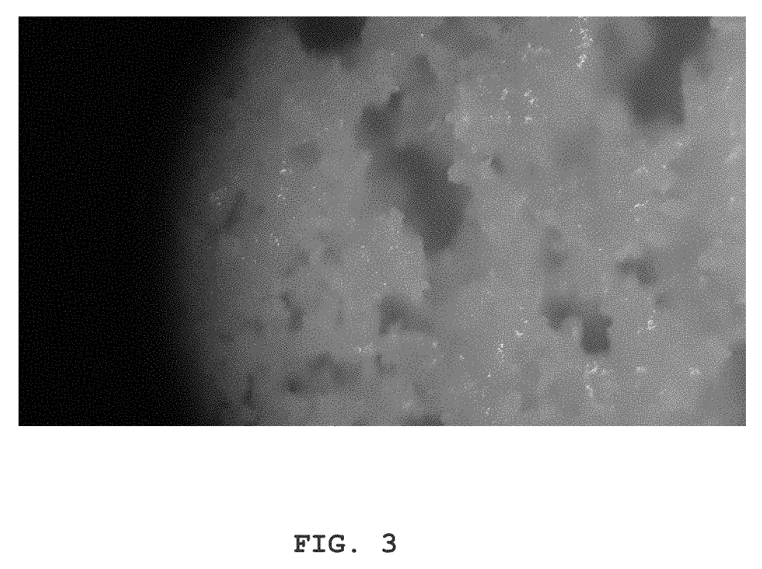

The powder coating formulation was applied as before. FIGS. 2 and 3 are photomicrographs that show a highly disrupted surface with cavities, holes and channels in the surface, which looks visually similar to the structure of trabecular bone. These features should provide an excellent substrate for bony ingrowth, osseointegration and strong adhesion of adjacent bone to the implant surface. Furthermore, since the surface contains significant hydrophilic charged zeolite, it should have a strong affinity for osteoblasts and bone cells and a resistance to formation of a fibrous apposition layer which occurs with PEEK. The elution of silver from the surface should be strongly enhanced compared to a flat surface, thereby enhancing the antimicrobial activity of the surface.

### Example 5

### Addition of Sodium Bicarbonate.

Sodium bicarbonate was added to a mixture of PEEK powder and silver zeolite to determine if the composition could be powder coated and to assess the surface morphology.

Composition

| Component | gm | (w/w) % |
|---|---|---|
| Peek Powder | 127 | 47.04 |
| Sodium Bicarbonate | 118 | 43.70 |
| Silver Zeolite | 25 | 9.26 |
| Total | 270 | 100 |

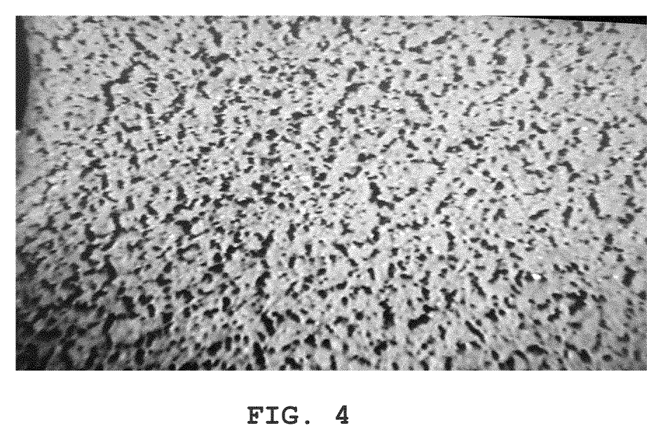

The material was applied as before and cured under the same conditions. A macrophotograph of the surface is presented in FIG. 4. Sodium bicarbonate partially decomposes at about 85 releasing carbon dioxide. It can be seen that a highly disrupted surface is produced here also.

What is claimed is:

1. A method of powder coating an implantable substrate, comprising:
creating a polymer formulation comprising a mixture of a polymer resin, zeolite and a micronized porogen;
providing an implantable substrate;
spray coating said polymer formulation onto said substrate; and
curing said coating on said substrate.

2. The method of claim 1, wherein said zeolite comprises antimicrobial metal ions.

3. The method of claim 2, wherein said ions are silver ions.

4. The method of claim 1, wherein said substrate is a hip stem.

5. The method of claim 1, wherein said substrate is a knee implant.

6. The method of claim 1, wherein said substrate is an intervertebral spacer.

7. The method of claim 1, wherein said substrate is a trauma plate.

8. The method of claim 1, further comprising extracting said porogen from said cured polymer formulation.

9. The method of claim 1, wherein said porogen is sodium chloride.

10. The method of claim 1, wherein said porogen is sodium bicarbonate.

11. The method of claim 1, wherein after said polymer and zeolite are mixed and spray coated on said substrate, said zeolite is post-loaded with metal ions.

12. The method of claim 1, wherein said polymer formulation further comprises carbon fibers.

13. A method of promoting osseointegration of an implant in a patient, comprising:
blending zeolite, a micronized porogen and a polyetheretherketone resin to form a polymer formulation, wherein the zeolite is blended into the resin in an amount effective to render the polyetheretherketone resin hydrophilic and impart a negative charge to said polyetheretherketone resin;
spray coating said polymer formulation onto an implant substrate;
curing said coating on said substrate;
extracting said porogen to form a network of pores in said coating; and
implanting the resulting implant into a patient in need thereof.

14. A surgical implant, comprising:
a metal substrate;
a spray-applied coating on said substrate, said spray-applied coating comprising polyetheretherketone and zeolite, wherein said coating has a network of pores formed by extraction of a micronized porogen from a blend of said polyetheretherketone, zeolite and micronized porogen after being spray-applied onto said metal substrate.
